(19) United States
(12) Patent Application Publication
(10) Pub. No.: US 2024/0226424 A1
(43) Pub. Date: Jul. 11, 2024

(54) APPLICATOR DEVICE FOR USER-WEARABLE INFUSION PUMP

(71) Applicant: Tandem Diabetes Care, Inc., San Diego, CA (US)

(72) Inventors: Neel Shah, San Diego, CA (US); Neal Johnston, San Diego, CA (US); Jacob Pearlman, San Diego, CA (US); Eduardo Ho, San Diego, CA (US); Tinh Tran, San Diego, CA (US)

(21) Appl. No.: 18/407,915

(22) Filed: Jan. 9, 2024

Related U.S. Application Data
(60) Provisional application No. 63/437,881, filed on Jan. 9, 2023.

(51) Int. Cl. A61M 5/142

(52) U.S. Cl. CPC A61M 5/14248; A61M 2005/14252; A61M 2205/121; A61M 2210/04

(57) ABSTRACT

Disclosed herein are systems and methods for attaching a user-wearable infusion pump system to a patient. In embodiments, an applicator device can be provided that can insert a cannula into the skin of the user for delivering medicament from the pump to the user and attach a pump holder or tray that holds the pump onto the skin of the user with a single user-initiated step.

### PRIORITY CLAIM

This application claims the benefit of U.S. Provisional Application No. 63/437,881 filed Jan. 9, 2023, which is hereby incorporated herein by reference in its entirety.

### TECHNICAL FIELD

The present disclosure relates to medical pumps for delivering medicament to a patient and, more specifically, to an applicator device for attaching a user-wearable pump to a user.

### BACKGROUND

There are many applications in academic, industrial, and medical fields that benefit from devices and methods that are capable of accurately and controllably delivering fluids, such as liquids and gases, that have a beneficial effect when administered in known and controlled quantities. Such devices and methods can be particularly useful in the medical field where treatments for many patients include the administration of a known amount of a substance at predetermined intervals.

One category of devices for delivering such fluids is that of pumps that have been developed for the administration of insulin and other medicaments for those suffering from both type I and type II diabetes. Some pumps configured as portable infusion devices can provide continuous subcutaneous medicament injection and/or infusion therapy for the treatment of diabetes. Such therapy may include, e.g., the regular and/or continuous injection or infusion of insulin into a person suffering from diabetes and offer an alternative to multiple daily injections of insulin by an insulin syringe or an insulin pen. Such pumps can be ambulatory/portable infusion pumps that are worn by the user and may use replaceable cartridges. Such pumps can deliver medicaments other than or in addition to insulin, such as glucagon. Examples of such pumps and various features that can be associated with such pumps include those disclosed in U.S. Pat. No. 8,287,495, which is hereby incorporated herein by reference in its entirety.

One type of pump that has been developed is a patch pump, or micro pump. Patch pumps generally are small pumps that are carried directly on the skin under the user's clothing. Many such pumps are situated directly on the infusion site such that no tubing is required to deliver the insulin and/or other medicament to the patient. Other patch pumps can be positioned on the patient's body with a short length of tubing extending to a nearby infusion site. Not unlike other types of pumps, but perhaps more typically, patch pumps can be at least in part disposable, meant to be worn for a period of time such as, e.g., a day or two, and then discarded and replaced by a new patch pump. Other patch pump designs contemplate a disposable component, such as a cartridge that contains medicament, and a reusable or durable component. In such configurations, the disposable and durable components may be joined together by the patient or caregiver in preparation for delivery of the medicament. Still other patch pumps may include a rechargeable durable component as well as a refillable and reusable medicament cartridge.

To put such patch pumps into use, a cannula for delivering medicament from the pump to the user must be inserted into the user's body and the pump must be adhered to the user's body. These operations can be difficult for a user to learn and/or carry out and can be prone to user error.

### SUMMARY

Disclosed herein are systems and methods for attaching a user-wearable infusion pump system to a patient. In embodiments, an applicator device can be provided that can insert a cannula into the skin of the user for delivering medicament from the pump to the user and attach a pump holder or tray that holds the pump onto the skin of the user with a single user-initiated step.

In embodiments, an applicator device for inserting a holder for a user-wearable infusion pump onto a body of a user can include a housing, a fluid path module including a cannula disposed within the housing and a drive wheel disposed within the housing. An actuation mechanism can be configured to selectively cause the drive wheel to be actuated. A tray release mechanism can be configured to releasably retain a tray configured to hold a user-wearable infusion pump on a body of a user. Actuation of the drive wheel can cause the drive wheel to rotate to insert the cannula into the user's skin and to release the tray from the tray release mechanism for application onto the user's skin.

In embodiments, an ambulatory infusion pump system can include a tray configured to hold a user-wearable infusion pump, an adhesive patch configured to adhere the tray to a body of a user and an applicator device for attaching the tray onto the body of the user. The applicator device can include a housing, a fluid path module including a cannula disposed within the housing and a drive wheel disposed within the housing. An actuation mechanism can be configured to selectively cause the drive wheel to be actuated. A tray release mechanism can be configured to releasably retain the tray. Actuation of the drive wheel can cause the drive wheel to rotate to insert the cannula into the user's skin and to release the tray from the tray release mechanism for application onto the user's skin.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict a patch pump system according to an embodiment of the disclosure.

FIGS. 2A-2B depict exploded views of an applicator device according to an embodiment of the disclosure.

FIGS. 3A-3D depict an applicator device according to an embodiment of the disclosure.

FIG. 4 depicts an applicator device according to an embodiment of the disclosure.

FIGS. 5A-5B depict cutaway views of an applicator device according to an embodiment of the disclosure.

FIGS. 6-6B depicts a cutaway view of an applicator device according to an embodiment of the disclosure.

FIG. 7 depicts a portion of a holder for a user-wearable infusion pump and a fluid path module according to an embodiment of the disclosure.

FIG. 8 depicts an applicator device according to an embodiment of the disclosure.

FIG. 9 depicts a portion of an applicator device and a holder for a user-wearable infusion pump according to an embodiment of the disclosure.

FIG. 10 depicts steps for applying a holder for a user-wearable infusion pump onto a body of a user with an applicator device according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

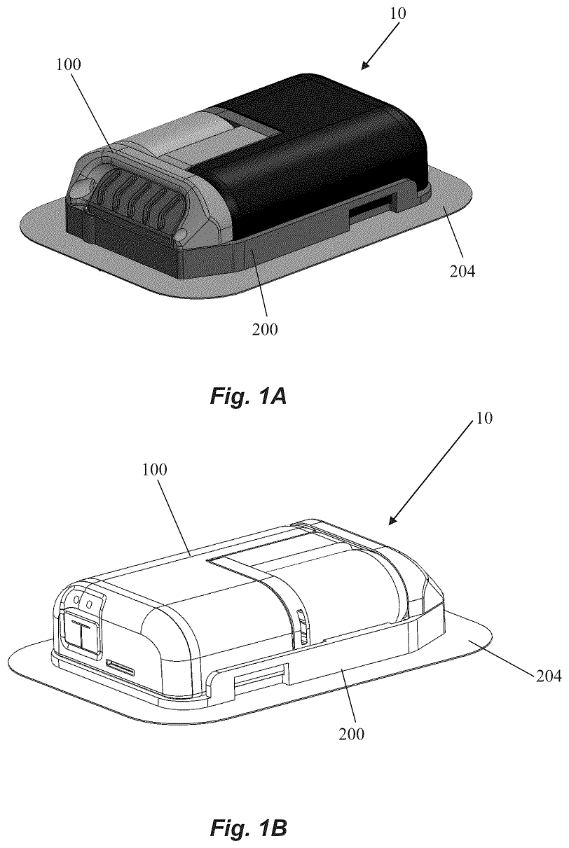

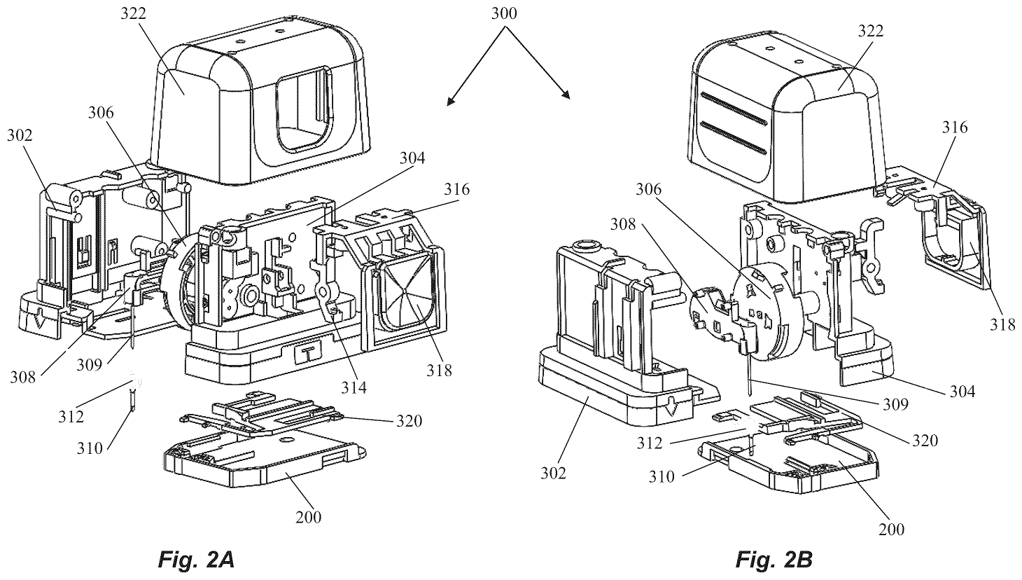

FIGS. 1A-1B and 2A-2B depict an ambulatory infusion pump system 10 according to an embodiment of the disclosure. System 10 can include an infusion pump 100 and a pump holder or tray 200 that can be used to releasably contain infusion pump 100 and that can be worn on a body of a user and attached thereto with an adhesive patch 204. Further details regarding such patch pump systems can be found in U.S. Provisional Patent Application No. 63/437,872 entitled USER-WEARABLE INFUSION PUMP SYSTEM, which is incorporated by reference herein in its entirety.

Referring now to FIGS. 2A-2B, the components of an applicator device 300 for attaching a patch pump system such as system 10 to a body of a user is depicted. In embodiments, applicator device 300 is used to attach a pump holder such as holder 200 to the user's body while also inserting the cannula 12 through which medicament is delivered to the user and the user subsequently inserts an infusion pump such as pump 100 onto tray (as further described in the application incorporated by reference above).

Applicator device 300 can include a backbone 302 and a drive wheel mount 304 configured to be attached to each outer to define a housing 303. Drive wheel 306 can be mounted to drive wheel mount 304 and interface with needle insertion hub 308. As will be discussed in more detail below, the needle insertion hub 308 can be operable to insert a cannula 310 of a fluid path module 312 into the skin of the user with a needle 309. A drive wheel release 314 configured to interface with the drive wheel 306 can be mounted to a drive frame 316 including a button 318. A tray release 320 configured to releasably retain a pump holder or tray 200 can be positioned at a distal end of the device 300. A cover 322 can be positioned around the internal components of the device and include an aperture 324 through which button 318 is accessible.

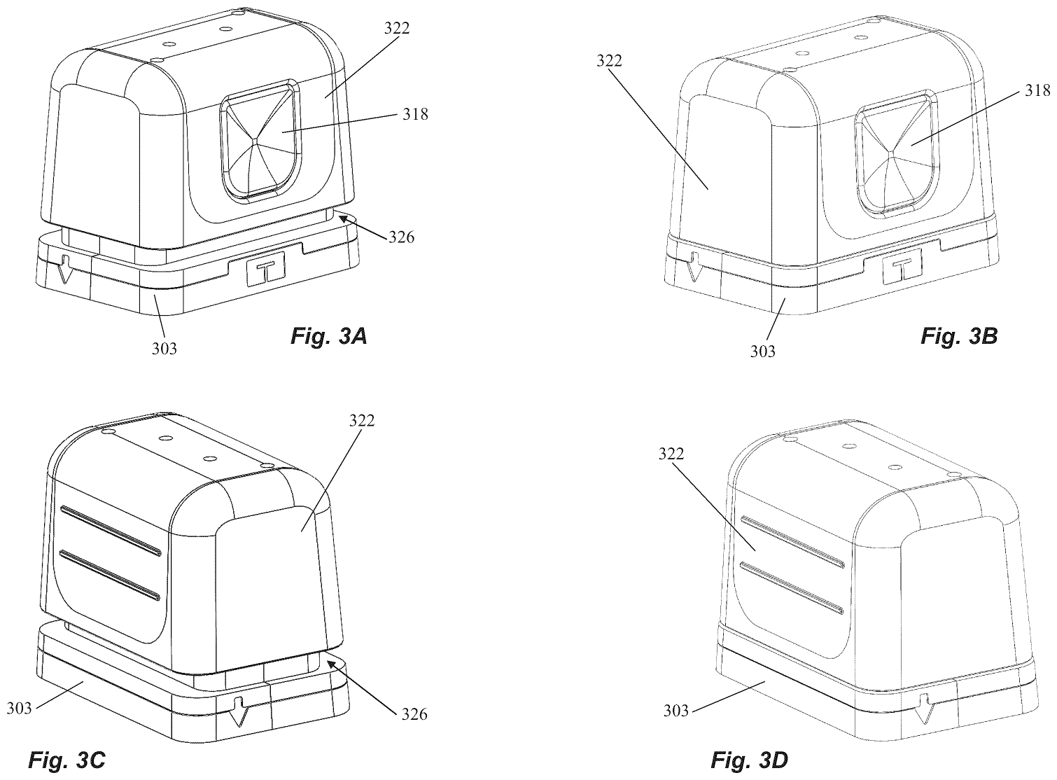

FIGS. 3A-3D depict views of an assembled applicator device 300. FIGS. 3A and 3C depict the device with a safety mechanism in place. The safety mechanism can be defined by the initial upward position of the cover being such that actuation of the button 318 does not release the drive wheel release. FIGS. 3B and 3D depict the device with the safety mechanism removed. In the position, the button 318 is aligned with the drive wheel release such that actuation of the button will actuation the drive wheel, as will be described in more detail below. A spring can be disposed within applicator that will cause the cover 322 to move back up to the upward position if the cover 322 is not held down prior to pressing the button 318. The user must therefore press down and hold the cover 322 to release the safety. The space 326 between the cover 322 and the housing 303 disappears when the safety is released. The device can then be actuated to deploy the cannula and tray with the button 318, as will be discussed on more detail below. After actuation, the spring within the housing is configured such that it will no longer automatically return the cover 322 to the upward position. Cover 322 therefore remains in the downward position of FIGS. 3B and 3D after deployment, providing a visual indication to the user that the applicator has been deployed.

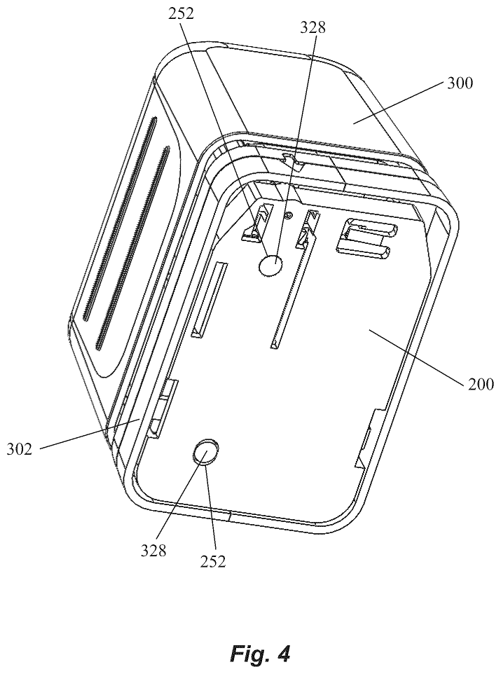

FIG. 4 depicts a bottom view of the assembled applicator device 300 including tray 200 releasably attached thereto. The bottom surface of backbone 302 includes a pair of pins 328 configured to be inserted through a pair of corresponding apertures 252 through tray 200 to ensure proper alignment of tray 200 in applicator 300.

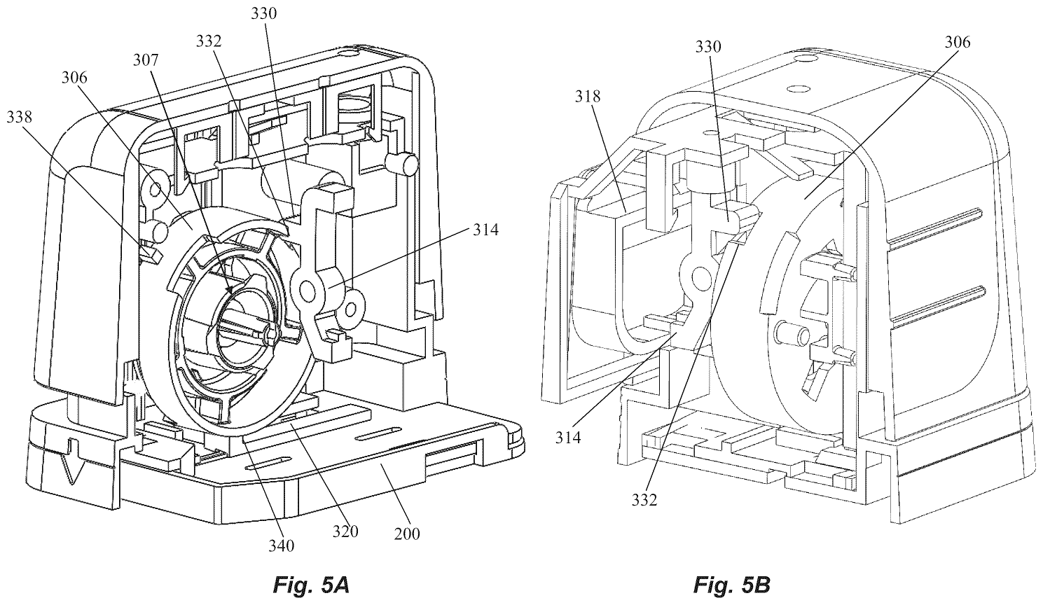

FIGS. 5A-7 depict further details regarding the operation of applicator device 300. Initially, and as shown in FIGS. 5A-5B, a projection 330 on drive wheel release 314 is disposed within a slot 332 in drive wheel 306, preventing the drive wheel 306 from rotating. Drive wheel 306 can be driven by a pre-wound torsion spring (not depicted) such that upon removal of the projection 330 from the slot 332 by pressing button 318 (with the safety mechanism released) the drive wheel 306 will automatically rotate. In an embodiment, torsion spring can be disposed in a cylindrical opening 307 in drive wheel 306 and be biased to rotate drive wheel 306 when not prevented from doing so by drive wheel release 314. Rotation of drive wheel causes the cannula 310 to be inserted into the skin of the user, the fluid path module 312 to be seated in the tray 200 and the tray 200 to be deployed from the applicator 300 onto the user.

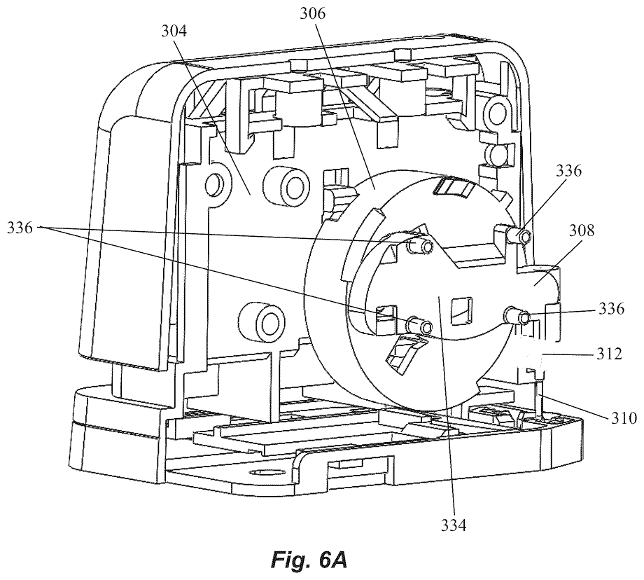

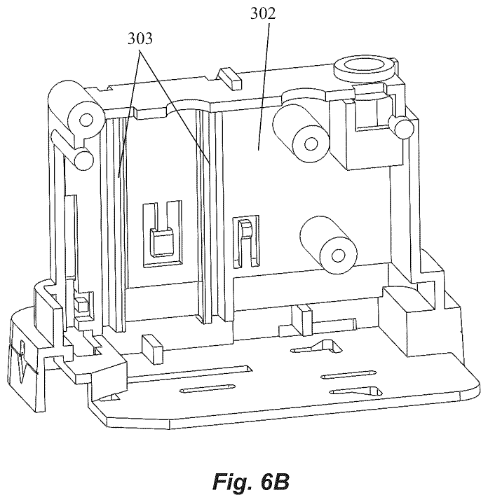

Referring to FIGS. 6A, upon initial rotation of drive wheel 306, linear slide 334 of insertion hub 308 inserts the cannula 310 into the user's body with needle 309 and seats the fluid path module 312 into a corresponding recess in the tray 200. Linear slide 334 can include guide pins 336 that interface with tracks 303 in backbone 302 to guide movement of the linear slide 334 as wheel 306 rotates. Tracks 303, as can be seen in FIG. 6B, can be a pair of parallel elongate slots configured such that upon initial rotation of drive wheel 306, the linear slide 334 moves down to insert the cannula 310 into the user's body with needle 309 and to seat the fluid path module 312 into a corresponding recess in the tray 200. Further rotation of guide wheel 306 causes the linear slide 334 to move back up to retract the needle 309 based on the guide tracks in backbone.

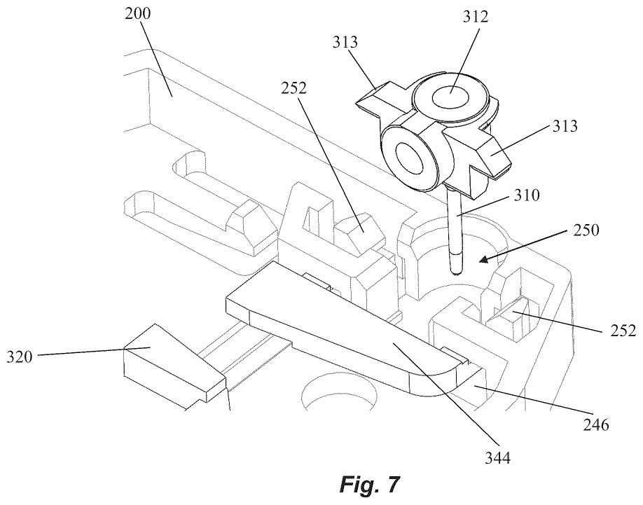

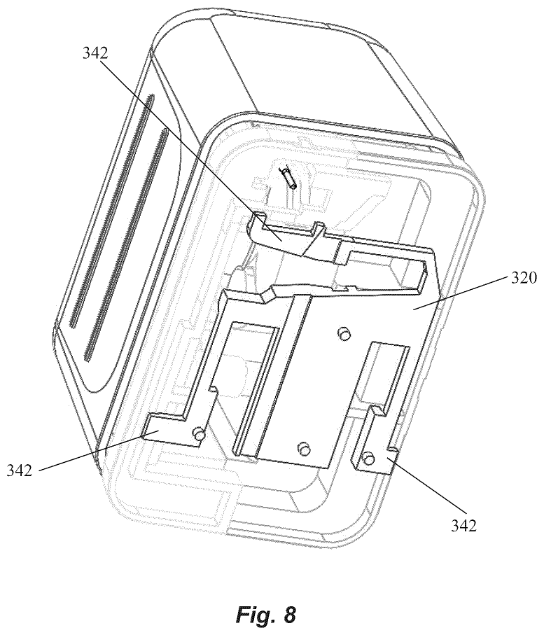

Further details regarding the insertion of fluid path module 312 into tray 200 can be seen in FIG. 7. Fluid path module 312 can include a pair of wings 313. Tray 200 can include an opening 250 for receiving the fluid path module 312 with a pair of projections or snaps 252 having angled surface on opposing sides of the opening 250. As the fluid path module 312 is lowered into the opening 250, wings 313 interface with snaps 252 and the fluid path module 312 is guided into opening 250 by the angled surfaces of snaps 252. As the wings 313 slide along the snaps 252, the snaps 252 flex outwardly to enable the fluid path module 312 to be inserted into the opening 250 and then snap back over the wings 313 once the fluid path module 312 is seated to firmly retain the fluid path module 312 in the tray 200. In embodiments, the fluid path module 312 is locked within and cannot be removed from the tray (without damaging the tray). The fluid path module is therefore disposed of when the tray is disposed of when the user rotates the infusion site and utilizes a new tray.

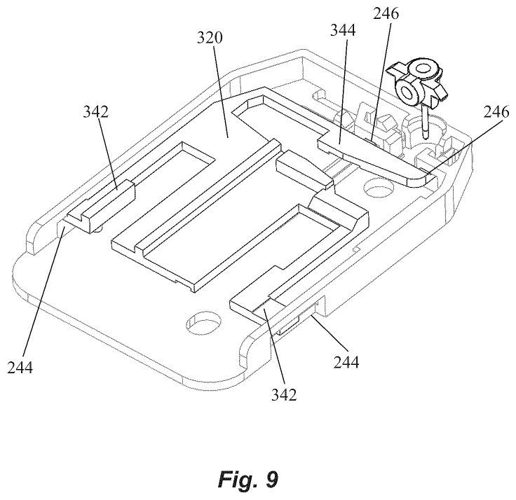

Following insertion of the cannula 310 and fluid path module 312 and retraction of the needle, further rotation of drive wheel 306 can release the tray 200 onto the body of the user. Initially, as shown in FIGS. 8-9, tray 200 is retained by tray release component 320 with arms 342, 344 of tray release component 320 having projections disposed in corresponding slots 244, 246 in tray 200. Referring again to FIGS. 5A-5B, as the drive wheel 306 is further rotated, a boss 338 on the drive wheel 306 will contact a ledge 340 on the tray release component 320. The causes the tray release component 320 to slide within the applicator device 300, which, in turn, causes arm 344 to slide back out of the corresponding tray slots 246 and arms 342 to flex out of the corresponding trays slots 244. This allows the tray 200 to separate from the tray release component 320 and inserter 300 and be applied to the user's body.

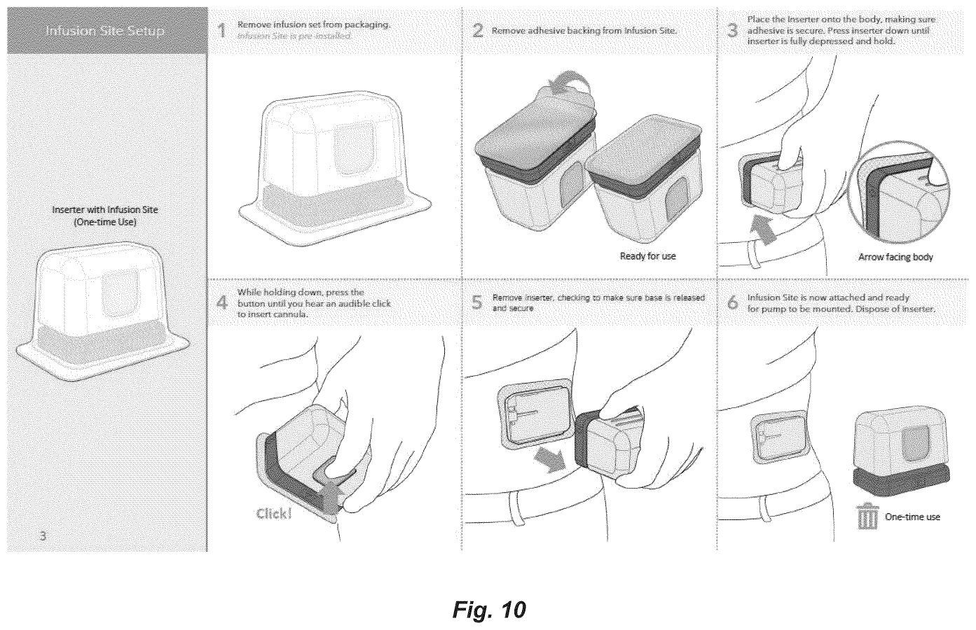

Steps for attaching a pump holder or tray 200 to a user using an applicator or inserter such as device 300 are depicted in FIG. 10. At step 1, the inserter and tray are removed from the packaging. An adhesive backing on the tray can then be removed at step 2 to expose an adhesive patch on the bottom of the tray. At step 3, the user places the inserter onto the body to adhere the adhesive patch onto the body and presses down and holds the inserter. With the inserter held down, the user presses the button of the inserter at step 4, which can result in an audible “click” sound as the cannula is inserted. The inserter can then be removed from the tray at step 5, with the tray remaining on the user's body. At step 6, the tray is attached and ready for the pump to be mounted thereon and the inserter can be disposed of.

In embodiments, an applicator device for inserting a holder for a user-wearable infusion pump onto a body of a user can include a housing, a fluid path module including a cannula disposed within the housing and a drive wheel disposed within the housing. An actuation mechanism can be configured to selectively cause the drive wheel to be actuated. A tray release mechanism can be configured to releasably retain a tray configured to hold a user-wearable infusion pump on a body of a user. Actuation of the drive wheel can cause the drive wheel to rotate to insert the cannula into the user's skin and to release the tray from the tray release mechanism for application onto the user's skin.

In some embodiments, a single actuation of the actuation mechanism causes both the cannula to be inserted into the user's skin and the tray release mechanism to release the tray.

In some embodiments, the applicator further comprises a cover disposed around and movable with respect to the housing and the actuation mechanism includes a button extending through the cover.

In some embodiments, the applicator further comprises a safety mechanism that prevents actuation of the button from actuating the drive wheel when the safety mechanism is engaged.

In some embodiments, when the cover is in an upright position with respect to the housing the safety mechanism prevents actuation of the button and if the cover is held down against the housing the safety mechanism is disengaged such that actuation of the button is enabled.

In some embodiments, the cover is biased to the upright position with respect to the housing prior to actuation of the drive wheel.

In some embodiments, following actuation of the drive wheel the cover is configured to remain held down against the housing.

In some embodiments, the tray release mechanism comprises one or more arms configured to be releasably inserted through one or more corresponding slots in the tray.

In some embodiments, the drive wheel is actuated by a pre-wound torsion spring.

In some embodiments, the applicator is configured as a one-time use device configured to be disposed of after a single actuation of the drive wheel.

In embodiments, an ambulatory infusion pump system can include a tray configured to hold a user-wearable infusion pump, an adhesive patch configured to adhere the tray to a body of a user and an applicator device for attaching the tray onto the body of the user. The applicator device can include a housing, a fluid path module including a cannula disposed within the housing and a drive wheel disposed within the housing. An actuation mechanism can be configured to selectively cause the drive wheel to be actuated. A tray release mechanism can be configured to releasably retain the tray. Actuation of the drive wheel can cause the drive wheel to rotate to insert the cannula into the user's skin and to release the tray from the tray release mechanism for application onto the user's skin.

In some embodiments, a single actuation of the actuation mechanism causes both the cannula to be inserted into the user's skin and the tray release mechanism to release the tray.

In some embodiments, the tray release mechanism comprises one or more arms configured to be releasably inserted through one or more corresponding slots in the tray.

In some embodiments, rotation of the drive wheel causes the one or more arms to disengage from the one or more corresponding slots to release the tray from the applicator device.

In some embodiments, at least one arm slides linearly out of a corresponding slot.

In some embodiments, at least one arm flexes to disengage from a corresponding slot.

In some embodiments, the fluid path module comprises a body having the cannula extending therefrom.

In some embodiments, actuation of the drive wheel causes the body of the fluid path module to be seated within a corresponding opening in the tray.

In some embodiments, the fluid path module and the opening include corresponding features configured to lock the fluid path module within the opening.

In some embodiments the system further comprises a user-wearable infusion pump configured to be releasably retained by the tray.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10,541,987; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; ; 2022/0037465; 2023/0040677; 2023/0047034; 2023/0113545 and 2023/0113755 and commonly owned U.S. patent applications Nos. 17/368,968; 17/896,492; 18/011,060; 18/071,814; 18/071,835; 18/075,029; 18/090,788 18/115,316; and 18/139,391.

For purposes of interpreting the claims, it is expressly intended that the provisions of 35 U.S.C. § 112(f) are not to be invoked unless the specific terms “means for” or “step for” are recited in a claim.

1. An applicator device for inserting a holder for a user-wearable infusion pump onto a body of a user, comprising:
a housing;
a fluid path module including a cannula disposed within the housing;
a drive wheel disposed within the housing;
an actuation mechanism configured to selectively cause the drive wheel to be actuated; and
a tray release mechanism configured to releasably retain a tray configured to hold a user-wearable infusion pump on a body of a user,
wherein actuation of the drive wheel causes the drive wheel to rotate to insert the cannula into the user's skin and to release the tray from the tray release mechanism for application onto the user's skin.

2. The applicator device of claim 1, wherein a single actuation of the actuation mechanism causes both the cannula to be inserted into the user's skin and the tray release mechanism to release the tray.

3. The applicator device of claim 1, further comprising a cover disposed around and movable with respect to the housing and wherein the actuation mechanism includes a button extending through the cover.

4. The applicator device of claim 3, further comprising a safety mechanism that prevents actuation of the button from actuating the drive wheel when the safety mechanism is engaged.

5. The applicator device of claim 4, wherein when the cover is in an upright position with respect to the housing the safety mechanism prevents actuation of the button and if the cover is held down against the housing the safety mechanism is disengaged such that actuation of the button is enabled.

6. The applicator device of claim 5, wherein the cover is biased to the upright position with respect to the housing prior to actuation of the drive wheel.

7. The applicator device of claim 6, wherein following actuation of the drive wheel the cover is configured to remain held down against the housing.

8. The applicator device of claim 1, wherein the tray release mechanism comprises one or more pins configured to be releasably inserted through one or more corresponding apertures in the tray.

9. The applicator device of claim 1, wherein the drive wheel is actuated by a pre-wound torsion spring.

10. The applicator device of claim 1, configured as a one-time use device configured to be disposed of after a single actuation of the drive wheel.

11. An ambulatory infusion pump system, comprising:
a tray configured to hold a user-wearable infusion pump;
an adhesive patch configured to adhere the tray to a body of a user; and
an applicator device for attaching the tray onto the body of the user, the applicator device comprising—
a housing;
a fluid path module including a cannula disposed within the housing;
a drive wheel disposed within the housing;
an actuation mechanism configured to selectively cause the drive wheel to be actuated; and
a tray release mechanism configured to releasably retain the tray,
wherein actuation of the drive wheel causes the drive wheel to rotate to insert the cannula into the user's skin and to release the tray from the tray release mechanism for application onto the user's skin.

12. The ambulatory infusion pump system of claim 11, wherein a single actuation of the actuation mechanism causes both the cannula to be inserted into the user's skin and the tray release mechanism to release the tray.

13. The ambulatory infusion pump system of claim 11, wherein the tray release mechanism comprises one or more arms configured to be releasably inserted through one or more corresponding slots in the tray.

14. The ambulatory infusion pump system of claim 13, wherein rotation of the drive wheel causes the one or more arms to disengage from the one or more corresponding slots to release the tray from the applicator device.

15. The ambulatory infusion pump system of claim 14, wherein at least one arm slides linearly out of a corresponding slot.

16. The ambulatory infusion pump system of claim 14, wherein at least one arm flexes to disengage from a corresponding slot.

17. The ambulatory infusion pump system of claim 11, wherein the fluid path module comprises a body having the cannula extending therefrom.

18. The ambulatory infusion pump system of claim 17, wherein actuation of the drive wheel causes the body of the fluid path module to be seated within a corresponding opening in the tray.

19. The ambulatory infusion pump system of claim 18, wherein the fluid path module and the opening include corresponding features configured to lock the fluid path module within the opening.

20. The ambulatory infusion pump system of claim 11, further comprising a user-wearable infusion pump configured to be releasably retained by the tray.
